(19) United States
(12) Patent Application Publication
(10) Pub. No.: US 2013/0056075 A1
(43) Pub. Date: Mar. 7, 2013

(54) COMPOUNDS FOR USE IN ELECTROLYTE FOR SOLAR CELL, METHOD FOR PREPARING THE SAME, AND ELECTROLYTE AND SOLAR CELL HAVING THE SAME

(71) Applicants: Yu-Hui Lee, Taoyuan (TW); Hsin-Ying Lu, Taoyuan (TW); Kuan-Wei Lee, Taoyuan (TW); Tzu-Heng Ko, Taoyuan (TW)

(72) Inventors: Yu-Hui Lee, Taoyuan (TW); Hsin-Ying Lu, Taoyuan (TW); Kuan-Wei Lee, Taoyuan (TW); Tzu-Heng Ko, Taoyuan (TW)

(73) Assignee: EVERLIGHT USA, INC., Pineville, NC (US)

(21) Appl. No.: 13/602,482

(22) Filed: Sep. 4, 2012

(30) Foreign Application Priority Data
Sep. 5, 2011 (TW) 100131874
Aug. 24, 2012 (TW) 101130772

(51) Int. Cl. C07D 403/12; C07D 235/06; H01G 9/022; H01L 51/46

(52) U.S. Cl. 136/263; 548/305.4; 548/310.1; 252/62.2

(57) ABSTRACT

Provided is a compound of formula (I):

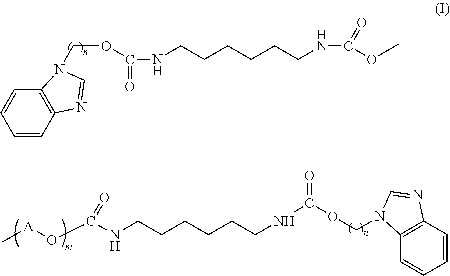

wherein A is C₂₋₃ alkylene; m is an integer ranging from 2 to 25; and n is an integer ranging from 3 to 10. An electrolyte for a dye-sensitized solar cell having the compound of formula (I) and/or a compound of formula (II) is further provided for increasing photoelectric conversion efficiency.

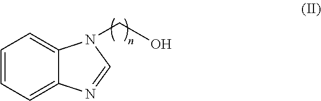

### BACKGROUND OF THE INVENTION

1. Field of the Invention

The present invention relates to compounds for use in electrolytes for solar cells, and more particularly, to a compound for use in an electrolyte for a dye-sensitized solar cell.

2. Description of Related Art

Solar energy is one of the energy sources that meet the energy need. A solar cell can directly convert solar energy into electrical energy, which not only resolves the global energy crisis, but also achieves the elimination of environmental pollutions. Generally, solar cells are classified into semiconductor solar cells such as silicon solar cells, and photoelectrochemical solar cells such as dye-sensitized solar cells (DSSC). Grätzel et al. have a series of publications (for example, O'Regan, B.; Grätzel, M. Nature 1991, 353, 737) on dye-sensitized solar cells in recent years. Dye-sensitized solar cells have advantages such as low production costs, light weights, flexibility, transparency, and easiness in being made into products with large areas. Therefore, dye-sensitized solar cells have various excellent properties, gradually making them highly prospective solar cells.

Generally speaking, a dye-sensitized solar cell includes cathode/anode electrodes, and the anode is formed by a conductive layer and a porous thin film formed with a porous material (such as titanium dioxide particles) on a substrate, wherein the porous thin film is coated with a photo-sensitive dye. Further, there is an electrolyte layer interposed between the anode and the cathode. As the photo-sensitive dye on the electrode absorbs sunlight, an electric potential difference is created, thereby generating an electric current. TW200810167 discloses a dye-sensitized solar cell having nano particles formed on a nano line to increase the contact area between the nano particles and the dye. TW200905939 discloses a dye-sensitized solar cell having improved cell performance by increasing electron injection efficiency. Moreover, TW201017955 discloses a gel electrolyte suitable for a dye-sensitized solar cell, to further decrease production cost of DSSC. TW201020295 discloses a dye compound having a high molar absorption coefficient. TW201036983 discloses a panchromatic photosensitizer complex having a better spectrum response and photo-electron conversion efficiency. TWM380573 discloses an improved electrode structure for enhancing dye absorption and absorption of solar energy by a dye-sensitized solar cell, and for inhibiting re-coupling of electrons and holes in a conductive unit, so as to increase the photo-electron conversion efficiency in the dye-sensitized solar cell. Moreover, Konkuk University of Korea has a publication in 2010 (Electrochimica Acta 55 (2010) 1483-1488), entitled “Synthesis of novel imidazolium-based electrolytes and application for dye-sensitized solar cells.” The publication discloses that an ionic compound, which results from the copolymerization of polyurea and an imidazolium-based compound, can be used in a dye-sensitized solar cell (the relevant patent thereof is published in 2011, KR10-2011-00011158). The ionic compound replaced a conventional electrolyte component, and a neutral precursor compound was not used in an electrolyte as an additive.

The dye-sensitized solar cells have poorer photo-electron conversion efficiency than silicon solar cells. However, the dye-sensitized solar cells can be produced at low cost. As such, dye-sensitized solar cells have the potential of becoming the major solar cells, if the photo-electron conversion efficiency thereof is improved. The electrode structure, dye and electrolyte are factors that affect the photo-electron conversion efficiency. Accordingly, it is an urgent issue in the industry of solar cells to improve the performance of dye-sensitized solar cells by controlling the above factors.

### SUMMARY OF THE INVENTION

The present invention provides a compound of formula (I):

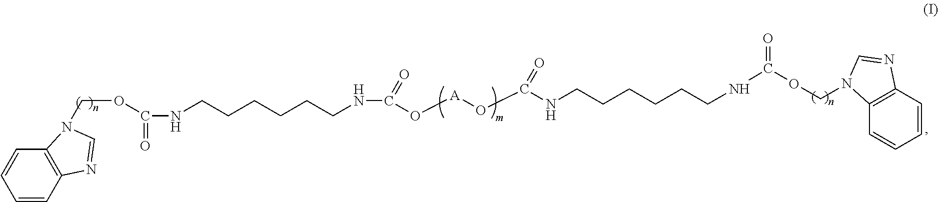

wherein A is C₂₋₃ alkylene; m is an integer ranging from 2 to 25; and n is an integer ranging from 3 to 10.

According to an embodiment of the present invention, A is ethylene, and m is an integer ranging from 2 to 25. According to an embodiment of the present invention, A is isopropylene, and m is an integer ranging from 2 to 15. According to an embodiment of the present invention, the compound of formula (I) is used in an electrolyte for a solar cell. According to an embodiment of the present application, the compound of formula (I) is used for preparing an electrolyte for a dye-sensitized solar cell.

The present invention further provides a compound of formula (II):

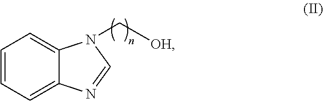

wherein n is an integer ranging from 3 to 10. According to an embodiment of the present invention, the compound of formula (II) is used for preparing a compound of formula (I). According to an embodiment of the present invention, the compound of formula (II) is used in an electrolyte for a solar cell. According to an embodiment of the present invention, the compound of formula (II) is used for preparing an electrolyte for a dye-sensitized solar cell.

The present invention also provides an electrolyte for a dye-sensitized solar cell, wherein the electrolyte includes a corn pound of formula (I) and/or a compound of formula (II).

The present invention further provides a dye-sensitized solar cell, including a substrate, a porous semiconductor film, a conductive film, an electrolyte and a dye compound, wherein the electrolyte includes a compound of formula (I) and/or a compound of formula (II).

The present invention further provides a method for preparing a compound of formula (I). The method includes the steps of performing a reaction of polyalkylene glycol, hexamethylene diisocyanate and a compound of formula (II).

According to an embodiment of the present invention, the method for preparing a compound of formula (I) includes the steps of performing a reaction of polyalkylene glycol and hexamethylene diisocyanate (HDI) to form a polyurethane intermediate, and performing a reaction of the polyurethane intermediate and a compound of formula (II). According to an embodiment of the present invention, the method for preparing a compound of formula (I) includes the steps of performing a reaction of hexamethylene diisocyanate and a compound of formula (II) to obtain an intermediate, and performing a reaction of the intermediate and polyalkylene glycol.

According to an embodiment of the present invention, the polyalkylene glycol is one of polyethylene glycol and polypropylene glycol.

The compounds of formulae (I) and (II) provided by the present invention can be used in an electrolyte for a dye-sensitized solar cell. According to an embodiment of the present invention, the compounds of formula (I) and/or formula (II) provided can be used as additives in an electrolyte for a dye-sensitized solar cell. The electrolyte having the compounds of formula (I) and/or formula (II) of the present invention may be used to prevent dark currents and facilitates an increase of open circuit voltage (V_{oc}). Moreover, the compounds of formulae (I) and formula (II) may be used to increase the photo-electron conversion efficacy of a dye-sensitized solar cell, which meets the industrial need.

### BRIEF DESCRIPTION OF THE DRAWINGS

The invention can be more fully understood by reading the following detailed description of the preferred embodiments, with reference made to the accompanying drawings, wherein:

FIGS. 1A and 1B show an ¹H-NMR spectrum and a GC-MS spectrum in Synthesis Example 1, respectively;

FIGS. 2A and 2B show an ¹H-NMR spectrum and a GC-MS spectrum in Synthesis Example 2, respectively;

FIGS. 3A and 3B show an ¹H-NMR spectrum and a GC-MS spectrum in Synthesis Example 3, respectively;

FIG. 4 shows an FTIR spectrum of hexamethylenediisocyanate (HDI);

FIG. 5 shows an FTIR spectrum in Example 1;

FIG. 6 shows an FTIR spectrum in Example 2;

FIG. 7 shows an FTIR spectrum in Example 3;

FIG. 8 shows an FTIR spectrum in Example 4; and

FIG. 9 shows an FTIR spectrum in Example 5.

### DETAILED DESCRIPTION OF THE PREFERRED EMBODIMENTS

The detailed description of the present invention is illustrated by the following specific examples. Persons skilled in the art can conceive the other advantages and effects of the present invention based on the disclosure of the specification of the present invention. The present invention can also be implemented and applied via different embodiments. Each of the details in the specification of the present invention can also be modified or altered without imparting from the spirit of the creation of the present invention, on the basis of different viewpoints and applications.

The term “weight average molecular weight” used herein is an Mw value of polystyrene, which is calculated by converting a measurement obtained by using tetrahydrofuran (THF) as a gel permeation chromatography (GPC) solvent.

The present invention provides a compound of formula (I):

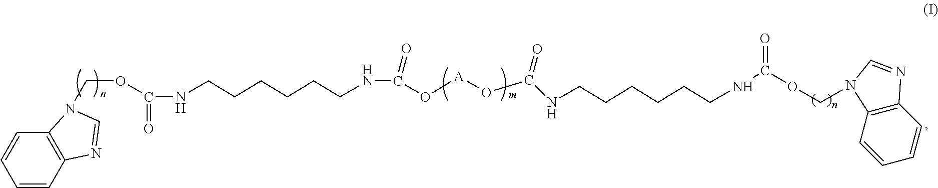

wherein A is C₂₋₃ alkylene; m is an integer ranging from 2 to 25; and n is an integer ranging from 3 to 10.

According to an embodiment of the present invention, A is ethylene, and m is an integer ranging from 2 to 25. In some of the aspects of such embodiment, m is an integer ranging from 3 to 20. In some of the aspects of such embodiment, m is an integer ranging from 5 to 20.

According to an embodiment of the present invention, A is isopropylene, and m is an integer ranging from 2 to 15. In some aspects of such embodiment, m is an integer ranging from 2 to 10.

According to the present, in formula (I), n is an integer ranging from 3 to 10, preferably ranging from 3 to 8, and more preferably ranging from 3 to 6.

According to an embodiment of the present invention, the compound of formula (I) may be added to an electrolyte for a solar cell, particularly, the electrolyte of a dye-sensitized solar cell.

According to an embodiment of the present invention, the compound of formula (I) is used in an electrolyte for a solar cell. According to an embodiment of the present invention, the compound of formula (I) is used for preparing an electrolyte for a dye-sensitized solar cell. According to an embodiment of the present invention, the compound of formula (I) can be used as an additive in an electrolyte for a dye-sensitized solar cell.

The present invention further provides a compound of formula (II):

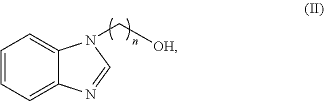

wherein n is an integer ranging from 3 to 10.

According to an embodiment of the present invention, n is preferably an integer ranging from 3 to 8, and more preferably ranging from 3 to 6.

According to an embodiment of the present invention, the compound of formula (II) can be used to prepare a compound of formula (I).

According to an embodiment of the present invention, the compound of formula (II) can be added to an electrolyte for a solar cell, particularly, an electrolyte for a dye-sensitized solar cell.

According to an embodiment of the present invention, the compound of formula (II) is used in an electrolyte for a solar cell. According to an embodiment of the present invention, the compound of formula (II) is used for preparing an electrolyte for a dye-sensitized solar cell. According to an embodiment of the present invention, the compound of formula (II) can be used as an additive in an electrolyte for a dye-sensitized solar cell.

According to an embodiment of the present invention, a compound of formula (I) is prepared by performing a reaction of polyalkylene glycol, hexamethylene diisocyanate and a compound of formula (II).

According to an embodiment of the present invention, the following method may be used to prepare a compound of formula (I): performing a reaction of polyalkylene glycol and hexamethylene diisocyanate to obtain a polyurethane intermediate, and performing a reaction of the polyurethane intermediate and a compound of formula (II).

Examples of polyalkylene glycol may be, but not limited to, polyethylene glycol and polypropylene glycol. According to an embodiment of the present invention, polyethylene glycol (PEG) is used for preparing a compound of formula (I), wherein the weight average molecular weight of polyethylene glycol is from 100 to 1000, preferably from 200 to 800, and more preferably from 300 to 600. According to an embodiment of the present invention, polypropylene glycol (PPG) is used for preparing a compound of formula (I), wherein the weight average molecular weight of polypropylene glycol is from 200 to 1000, preferably from 200 to 800, and more preferably from 200 to 600. The reaction of the polyalkylene glycol and hexamethylene diisocyanate to form a polyurethane intermediate is performed normally at 80 to 95° C. for 2 to 4 hours.

After polyalkylene glycol is reacted with hexamethylene diisocyanate (HDI) to obtain a polyurethane intermediate, the reaction of the polyurethane intermediate and a compound of formula (II) is performed at 80 to 95° C. for 2 to 4 hours.

According to another embodiment of the present invention, a compound of formula (I) may be prepared by performing a reaction of hexamethylene diisocyanate (HDI) and a compound of formula (II) to obtain an intermediate, and then performing a reaction of the intermediate and polyalkylene glycol.

According to the present invention, the reaction of benzimidazole and a compound of formula (III) is performed to form the compound of formula (II):

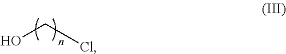

wherein n is an integer ranging from 3 to 10.

In an embodiment of the present, n is an integer preferably ranging from 3 to 8, and more preferably ranging from 3 to 6.

The reaction is usually performed in the presence of a solvent. The solvent may be a common solvent in the art, and there is no particular limitations. One or more solvents may be used, and when a mixture of two or more solvents are used, there are no specific limitations to the mixing ratio.

The solvent may be, but not limited to, toluene or dimethyl formamide (DMF). One or more solvents may be used, and when a mixture of two or more solvents are used, there are no specific limitations to the mixing ratio.

The reaction is usually performed in the presence of an alkali.

The alkali may be, but not limited to, potassium tert-butoxide, sodium hydroxide (NaOH) or potassium hydroxide (KOH).

The compounds of formulae (I) or (II) of the present invention may be added to an electrolyte for a solar cell, particularly an electrolyte for a dye-sensitized solar cell.

The present invention further includes an electrolyte for a dye-sensitized solar cell.

According to an embodiment of the present invention, the electrolyte includes a salt selected from metal iodide, an imidazolium iodide salt derivative or a salt of a combination thereof; iodine; guanidine thiocyanate; compounds of formula (I) and/or (II) (as mentioned previously); and a solvent.

The amount of metal iodide, an imidazolium iodide salt derivative or a salt of a combination thereof is from 1 to 20 wt %, based on the total weight of the electrolyte.

Metal iodide may be, but not limited to, potassium iodide, lithium iodide, sodium iodide or a combination thereof. Preferably, metal iodide is lithium iodide, sodium iodide or a combination thereof.

The imidazolium iodide salt derivative may be, but not limited to, 1-methyl-3-propyl imidazolium iodide (PMII), 1,3-dimethylimidazolium iodide, 1-methyl-3-ethyl imidazolium iodide, 1-methyl-3-butylimidazolium iodide, 1-methyl-3-pentyl-imidazolium iodide, 1-methyl-3-hexylimidazolium iodide, 1-methyl-3-heptylimidazolium iodide, 1-methyl-3-octylimidazolium iodide, 1,3-diethylimidazolium iodide, 1-ethyl-3-propylimidazolium iodide, 1-ethyl-3-butylimidazolium iodide, 1,3-propylimidazolium iodide, 1-propyl-3-butylimidazolium iodide, or a combination thereof. Preferably, the imidazolium iodide salt derivative may be 1-methyl-3-propylimidazolium iodide, 1-methyl-3-ethylimidazolium iodide, 1-methyl-3-butylimidazolium iodide, 1-methyl-3-pentyl-imidazolium iodide, 1,3-diethylimidazolium iodide, 1-ethyl-3-propylimidazolium iodide, or a combination thereof. One or more imidazolium iodide salt derivatives may be used, and when a mixture of two or more imidazolium iodide salt derivatives are used, there are no specific limitations to the mixing ratio.

The amount of iodine is from 1 to 3 wt %, based on the total weight of the electrolyte.

The amount of guanidine thiocyanate (GuNCS) is from 1 to 3 wt %, based on the total weight of the electrolyte.

The amount of the compounds of formulae (I) or (II) is from 8 to 85 wt %, based on the total weight of the electrolyte.

The amount of the solvent is from 5 to 80 wt %, based on the total weight of the electrolyte.

The solvents for use in an electrolyte for a dye-sensitized solar cell may be, but not limited to, acetonitrile, 3-methoxyl-propionitrile (3-MPN), N-methyl-2-pyrrolidone (NMP), propylene carbonate or γ-butyrolactone. One or more of the above solvents may be used, and when a mixture of two or more solvents are used, there are no specific limitations to the mixing ratio.

According to an embodiment of the present invention, other additives may be optionally added to the electrolyte for a dye-sensitized solar cell. The additives may be, but not limited to, an organic amine hydroiodide, a benzimidazole derivative, a pyridine derivative, or a combination thereof.

The organic amine hydroiodide may be, but not limited to, triethylamine hydroiodide (THI), tripropylamine hydroiodide, tributylamine hydroiodide, tripentylamine hydroiodide, trihexylamine hydroiodide or a combination thereof. Preferably, the organic amine hydroiodide may be triethylamine hydroiodide (THI), tripropylamine hydroiodide, tributylamine hydroiodide or a combination thereof. More preferably, the organic amine hydroiodide is triethylamine hydroiodide. One or more organic amine hydroiodides may be used, and when a mixture of two or more organic amine hydroiodides are used there are no specific limitations to the mixing ratios.

The benzimidazole derivative and the pyridine derivative may be, but not limited to, N-methylbenzimidazole (NMBI), N-butylbenzimidazole (NBB), 4-tert-butylpyridine (4-TBP) or a combination thereof. One or more benzimidazole derivatives and/or pyridine derivatives may be used, and when a mixture of two or more benzimidazole derivatives and pyridine derivatives are used, there are no specific limitations to the mixing ratio.

The above electrolyte may be used for preparing a dye-sensitized solar cell.

According to an embodiment of the present invention, the dye-sensitized solar cell includes a photoanode having a dye compound; a cathode; and an electrolyte layer interposed between the photoanode and the cathode. According to an embodiment of the present invention, the electrolyte layer is formed on the surface of the cathode which is in contact with the photoanode. According to an embodiment of the present invention, the dye-sensitized solar cell includes a substrate, a porous semiconductor film, a conductive film, an electrolyte and a dye compound.

According to an embodiment of the present invention, the electrolyte for a dye-sensitized solar cell includes compounds of formula (I) and/or formula (II). According to an embodiment of the present invention, the compound of formula (I) and/or formula (II) can be used as electrolyte additives of a dye-sensitized solar cell.

The fabrication of a dye-sensitized solar cell may be done using a conventional method in the art, and there are no particular limitations.

Generally speaking, the substrate is a transparent substrate. There are no specific limitations to the material of the transparent substrate, as long as it is a transparent material capable of blocking the entry of moisture or gases from the outside of the dye-sensitized solar cell, and having solvent and weather tolerance. The transparent substrate includes, but not limited to: substrates made from transparent inorganic materials, such as a quartz substrate, a glass substrate; and transparent plastic substrate, such as a polyethylene terephthalate (PET) substrate, a poly(ethylene naphthalene-2,6-dicarboxylate (PEN) substrate, a polycarbonate (PC) substrate, a polyethylene (PE) substrate, a polypropylene (PP) substrate, and a polyimide (PI) substrate. Preferably, the material of the transparent substrate is glass. Moreover, The thickness of the transparent substrate is not particularly limited, and can be designed based on transmittance and properties of the dye-sensitized solar cell.

In the dye-sensitized solar cell, the porous semiconductor film may be formed by semiconductor nanoparticles. Suitable semiconductor nanoparticles may include: silicon, titanium dioxide, tin dioxide, zinc oxide, tungsten trioxide, niobium pentoxide, strontium titanium trioxide or a combination thereof. Preferably, the semiconductor nanoparticles are titanium dioxide nanoparticles. Usually, the average diameter of the semiconductor nanoparticles is from 5 to 500 nm, and preferably from 10 to 50 nm. The thickness of the porous semiconductor film is from 5 to 25 μm. In the dye-sensitized solar cell, there may be one or more layers of the porous semiconductor film. During the making of the porous semiconductor film, the semiconductor nanoparticles with various diameters are used. In other words, each of the layers has a different diameter of the semiconductor nanoparticles. For example, the semiconductor nanoparticles with diameters of from 5 to 50 nm are first coated to form a coated thickness of from 5 to 20 μm, and then the semiconductor nanoparticles with diameters of from 200 to 400 nm are coated to form a coated thickness of from 3 to 5 μm.

According to the present invention, there are no particular limitations to the method for fabricating a photoanode, and a conventional method in the art may be used. However, if the porous semiconductor film in the dye-sensitized solar cell is formed by semiconductor nanoparticles, the semiconductor nanoparticles are first made into a paste, which is to be coated on a transparent substrate (for example, by blade coating, screen printing, spin coating, spray coating or wet coating, or typical wet coating, but there are no specific limitations) to form a photoanode. In addition, coating may be performed once or multiple times to reach a desired thickness.

Generally speaking, a transparent conductive film is used. material of the conductive film may be an oxide material selected from indium tin oxide (ITO), fluorine-doped tin oxide (FTO), zinc oxide-gallium oxide (ZnO—Ga₂O₃,) zinc oxide-aluminum oxide (ZnO-Al₂O₃), and or tin-based oxides.

The dye compound is disposed on the conductive film and filled in pores of the porous semiconductor film. The dye compound may be a common dye compound in the art, and there are no particular limitations. In the fabrication of a dye-sensitized solar cell, the dye compound may be dissolved in a suitable solvent to form a dye solution. The solvent may be, but not limited to, acetonitrile, methanol, ethanol, propanol, butanol (such as tert-butyl alcohol), dimethyl formamide, N-methylpyrrolidone or a mixture thereof. In the fabrication of a dye-sensitized solar cell, the transparent substrate coated with the porous semiconductor film is dipped in the dye solution, so as to make the dye compound in the dye solution is adsorbed by the porous semiconductor film.

The material of the cathode in the dye-sensitized solar cell may be any conductive material, and there are no particular limitations. Alternatively, the material of the cathode may be an insulating material, as long as the cathode has a transmissible layer with transmission formed on the surface thereof facing the photoanode. Usually, a substance with electrochemical stability, which may be, but not limited to, platinum, gold, carbon and the like may be used for forming a cathode.

The electrolyte layer is formed between the cathode and the porous semiconductor film. The above-mentioned electrolyte may be used for preparing a dye-sensitized solar cell.

According to an embodiment of the present invention, the dye compound may be applied on a substrate having a conductive film and a porous semiconductor film thereon to prepare a photoanode. Further, after forming the cathode, an electrolyte is injected to prepare a dye-sensitized solar cell.

The present invention is more specifically illustrated by, but not limited to, the following examples which are not intended to limit the scope of the present invention. Unless otherwise specified, “%” or “weight part(s)” for expressing the amount of any component or substance in the following examples and comparative examples is based on weight.

### EXAMPLES

### Preparation of a Compound of Formula (II)

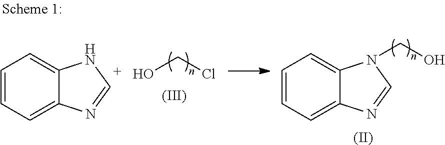

### Synthesis Example 1

### 1-benzimidazolepropanol (compound (IIa))

An amount of 14.176 g of benzimidazole (0.12 mol) and 14.80 g of potassium tert-butoxide (0.132 mol) were placed in a three-necked flask, and then 85 ml of dimethyl sulfoxide (DMSO) was added and stirred for 1 hour until dissolution. Then, 14.69 g of 1-chloro-3-hydroxypropane (0.15 mol) was dropped slowly into the three-necked flask using a 50 ml burette at 60° C. under nitrogen, and stirred for 6 hours. After the reaction was completed, 250 ml of ethyl acetate and 250 ml of water were added thereto for extraction, and the step was repeated for three times. An organic layer was dried with anhydrous magnesium sulfate (MgSO₄). The solvent used after filtering magnesium sulfate off was removed by using a rotary concentrator, and then the resultant impure solid was re-crystallized with ethyl acetate for purification. The organic solvent was removed by using the rotary concentrator to obtain compound (IIa) (17.3 g, 0.098 mol, yield of 74%).

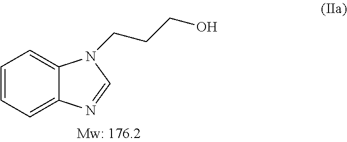 [SMILES: OCCCn1cnc2ccccc21]

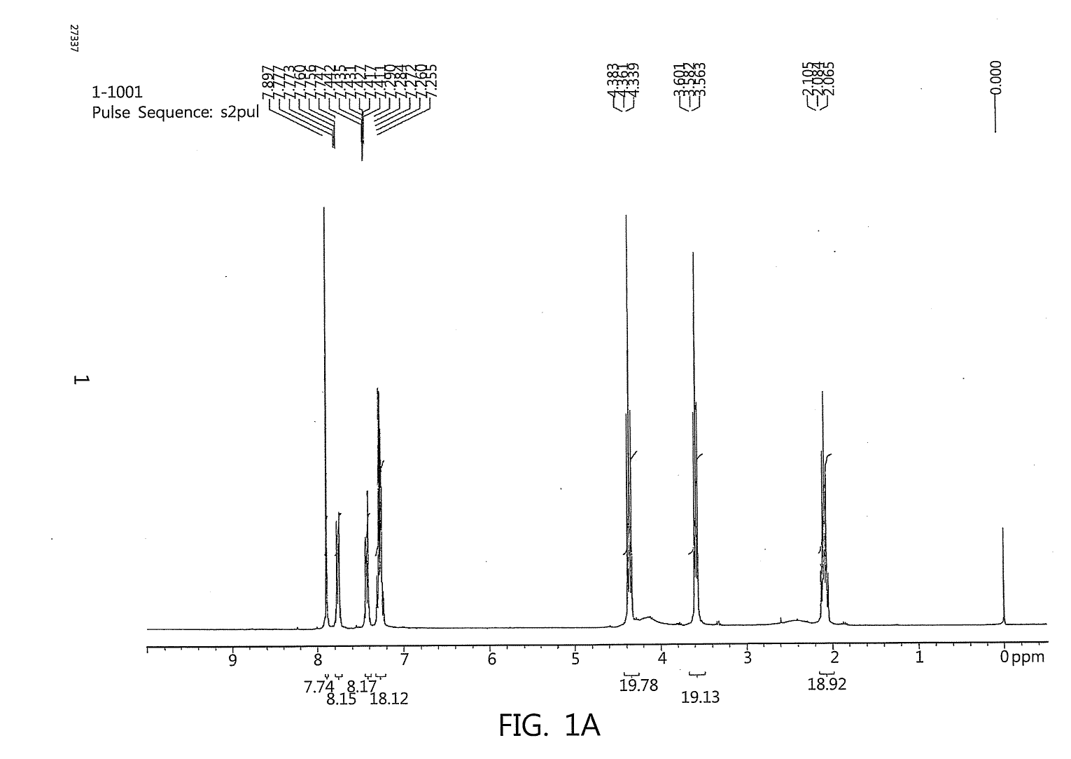

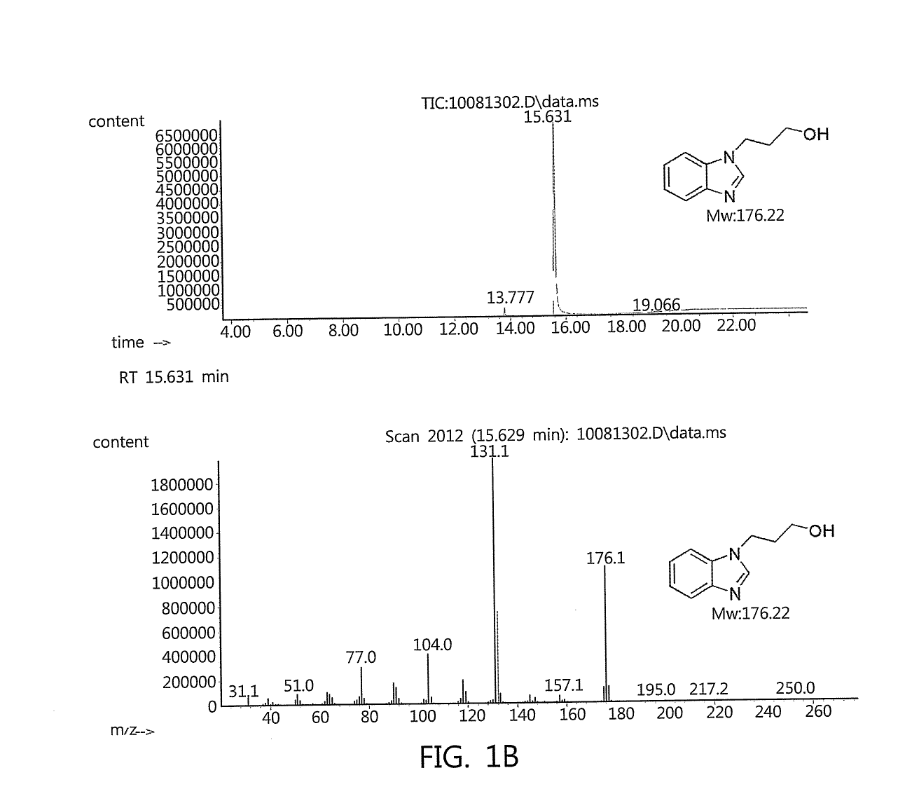

FIGS. 1A and 1B show an ¹H-NMR spectrum and a GC-MS spectrum, respectively.

The test conditions for the GC-MS test are as follows.

| | |
|---|---|
| GC/MS | 6890N/5975B type |
| Column | DB-5MS 30 m × 0.25 mm × 0.25 um |
| Temperature of oven | 60□/3 min 15□/min 310□/5 min |
| Injection temperature | 260□ |
| Test temperature | 300□ |
| Flow rate | 1.0 mL/min |
| Injection volume | 1 uL |
| Splitless | 0 |
| Mass range | 30 to 550 |
| Prepared solvent | CH₃OH |
| Solvent delay | 3.6 min |

¹H-NMR: (300 MHz, CDCl₃, ppm): δ=7.90 (s, 1H), 7.76 (dd, J=1.2. 1.2 Hz, 1H), 7.44 (dd, J=1.2. 1.2 Hz, 1H), 7.29-7.26 (m, 2H), 4.36 (t, J=6.6 Hz, 2H), 3.58 (t, J=5.7 Hz, 2H), 2.11-2.07 (m, 2H).

GC-MS (m/z): 176.22 calcd. 176.1 found.

### Synthesis Example 2

### 1-benzimidazolebutanol (compound IIb)

An amount of 20 g of benzimidazole (0.169 mol) and 21.32 g of potassium tert-butoxide (0.19 mol) were placed in a three-necked flask, and then 130 ml of dimethyl sulfoxide (DMSO) was added thereto and stirred for 1 hour until dissolution. Then, 25.5 g of 4-chloro-1-butanol (0.23 mol) was slowly dropped to the three-necked flask using a 50 ml burette at 60° C. under nitrogen, and stirred for 6 hours. After the reaction was completed, 250 ml of ethyl acetate and 250 ml of water were added thereto for extraction, and the step was repeated for three times. An organic layer was dried with anhydrous magnesium sulfate (MgSO₄). The solvent used after filtering magnesium sulfate off was removed by using a rotary concentrator. After the concentration, the resultant impure solid was purified by using column chromatography (ethyl acetate/methanol, 98:2, R_{f}=0.4). Then, the organic solvent was removed by the rotary concentrator, to obtain compound (IIb) (13.8 g, 0.072 mol, yield of 38%).

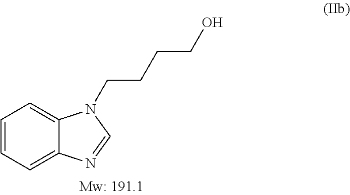 [SMILES: OCCCCn1cnc2ccccc21]

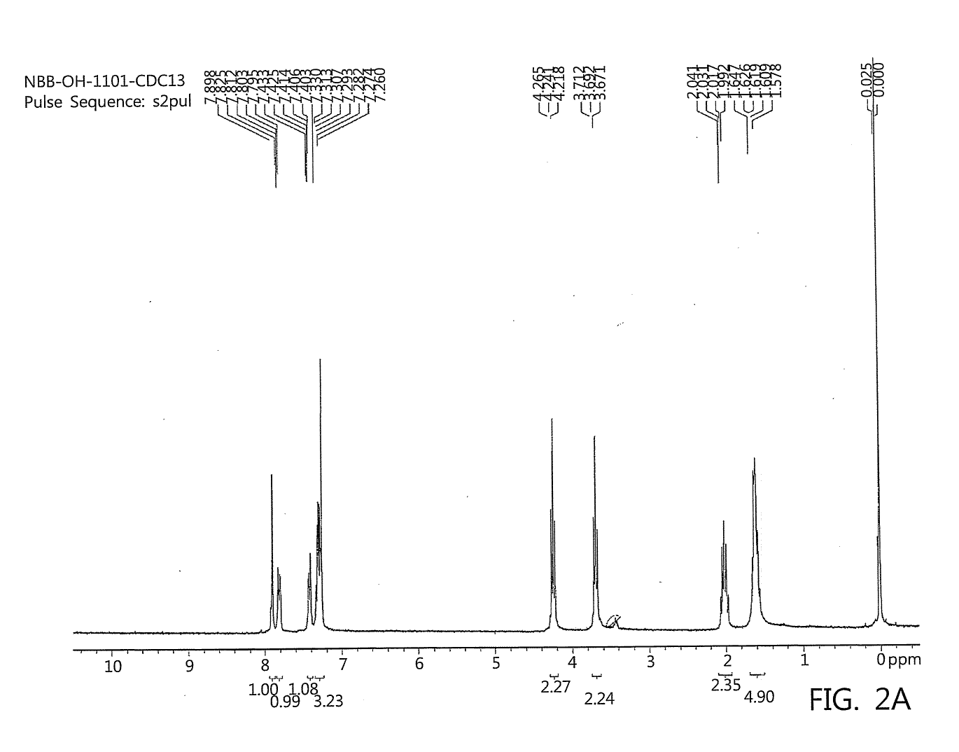

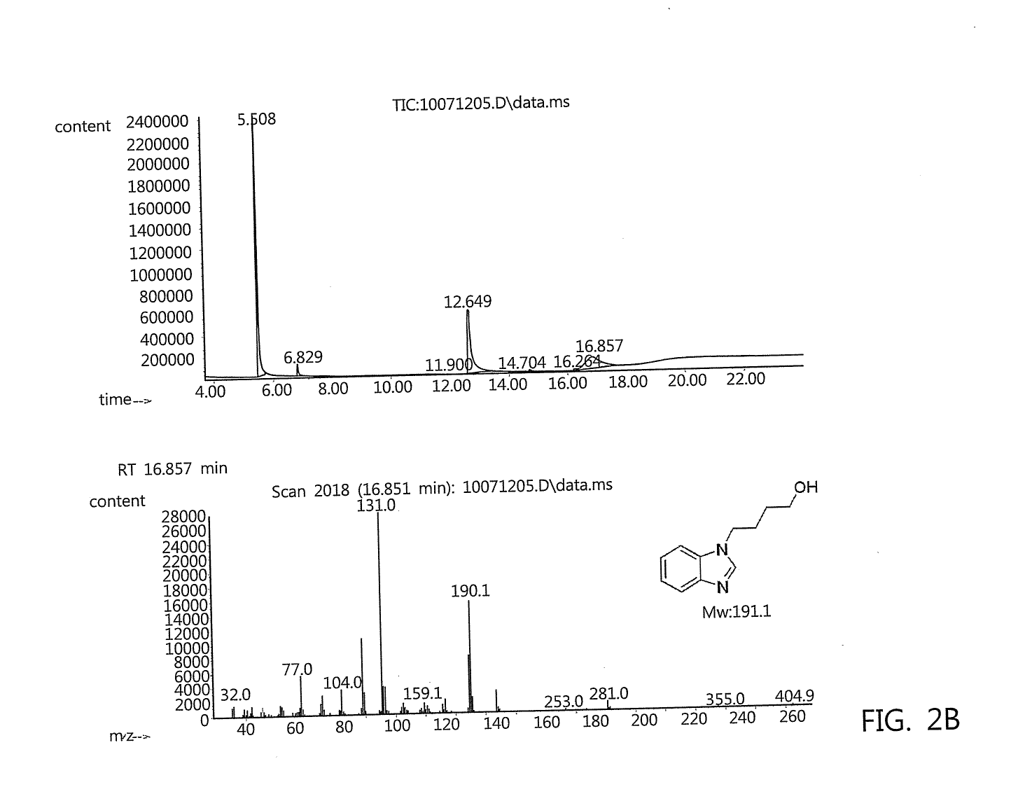

FIGS. 2A and. 2B show an ¹H-NMR spectrum and a GC-MS spectrum, respectively. The test conditions for the GC-MS test are as follows.

| | |
|---|---|
| GC/MS | 6890N/5975B type |
| Column | DB-5MS 30 m × 0.25 mm × 0.25 um |
| Temperature of oven | 60□/3 min 15□/min 300□/5 min |
| Injection temperature | 260□ |
| Test temperature | 300□ |
| Flow rate | 1.0 mL/min |
| Injection volume | 1 uL |
| Splitless | 0 |
| Mass range | 30 to 550 |
| Prepared solvent | CH₃OH |
| Solvent delay | 3.6 min |

¹H-NMR: (300 MHz, CDCl₃, ppm): δ=7.90 (s, 1H), 7.80 (dd, J=2.4. 2.4 Hz, 1H), 7.41 (dd, J=2.4. 2.4 Hz, 1H), 7.31-7.26 (m, 2H), 4.24 (t, J=7.2 Hz, 2H), 3.69 (t, J=6 Hz, 2H), 2.04-1.99 (m, 2H), 1.64-1.58 (m, 2H).

GC-MS (m/z): 190.1 calcd. 190.1 found.

### Synthesis Example 3

### 1-benzimidazolehexanol (compound (IIc))

An amount of 20 g of benzimidazole (0.169 mol) and 22.45 g of potassium tert-butoxide (0.2 mol) were placed in a three-necked flask, and then 85 ml of dimethyl sulfoxide (DMSO) was added thereto and stirred for 1 hour until dissolution. Then, 20 g of 6-chloro-1-hexanol (0.2 mol) was slowly dropped to the three-necked flask using a 50 ml burette at 60° C. under nitrogen, and stirred for 6 hours. After the reaction was completed, 250 ml of ethyl acetate and 250 ml of water were added thereto for extraction, and the step was repeated for three times. An organic layer was dried with anhydrous magnesium sulfate (MgSO₄). The solvent used after filtering magnesium sulfate off was removed by using a rotary concentrator. After the concentration, the resultant impure solid was purified by using column chromatography (ethyl acetate/methanol, 98:2, R_{f}=0.4). Then, the organic solvent was removed by using the rotary concentrator, to obtain compound (IIc) (28.46 g, 0.130 mol, yield: 77%).

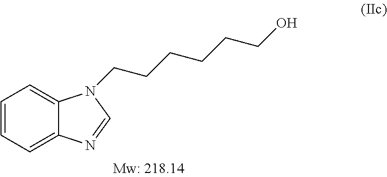 [SMILES: OCCCCCCn1cnc2ccccc21]

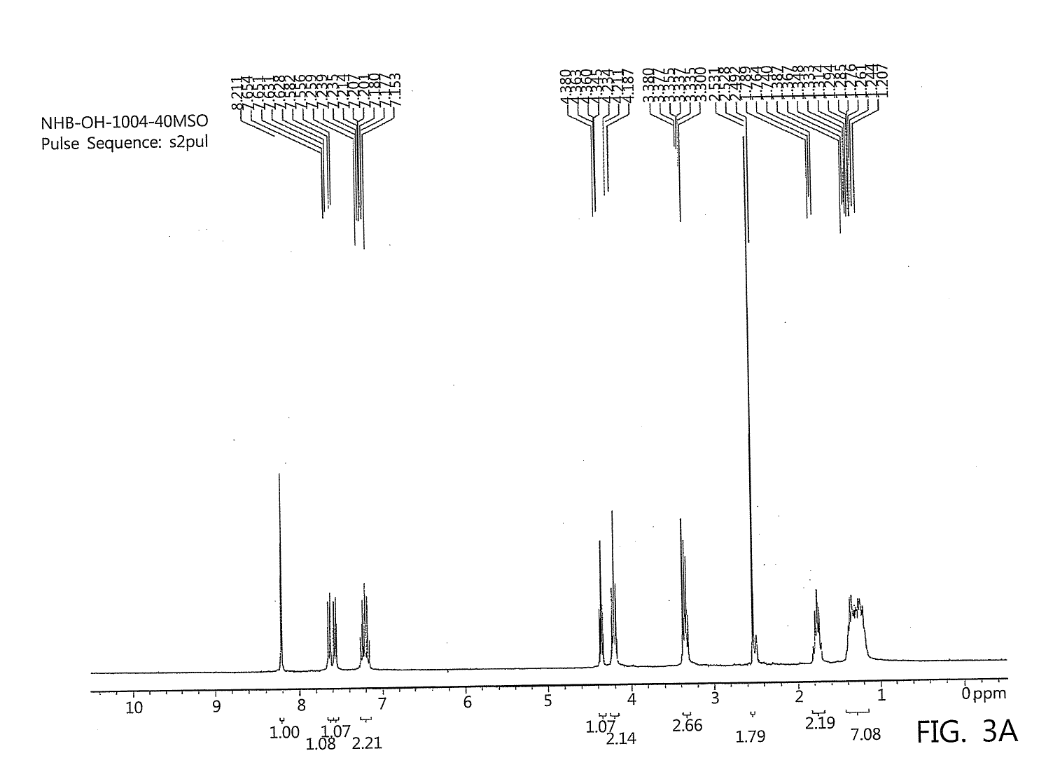

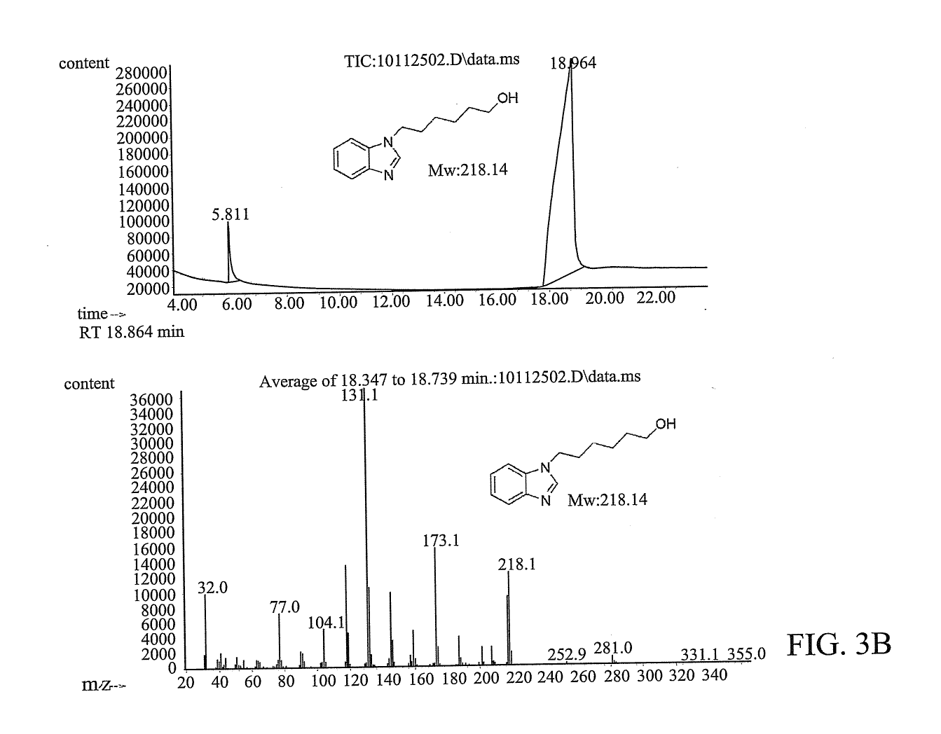

FIGS. 3A and 3B show an ¹H-NMR spectrum and a GC-MS spectrum, respectively. The test conditions for the GC-MS test are as follows.

| | |
|---|---|
| GC/MS | 6890N/5975B type |
| Column | DB-5MS 30 m × 0.25 mm × 0.25 um |
| Temperature of oven | 60□/3 min 15□/min 300□/5 min |
| Injection temperature | 260□ |
| Test temperature | 300□ |
| Flow rate | 1.0 mL/min |
| Injection volume | 1uL |
| Splitless | 0 |
| Mass range | 30 to 550 |
| Prepared solvent | CH₃OH |
| Solvent delay | 3.6 min |

¹H-NMR: (300 MHz, DMSO, ppm): δ=8.21 (s, 1H), 7.65 (dd, J=0.9. 0.9 Hz, 1H), 7.58 (d, J=7.8 Hz, 1H), 7.26-7.15 (m, 2H), 4.38-4.36 (m, 1H), 4.21 (t, J=6.9 Hz, 2H), 3.34-3.33 (m, 2H), 1.79-1.74 (m, 2H), 1.39-1.21 (m, 4H).

GC-MS (m/z): 218.14 calcd. 218.1 found.

### Preparation of a Compound of Formula (I)

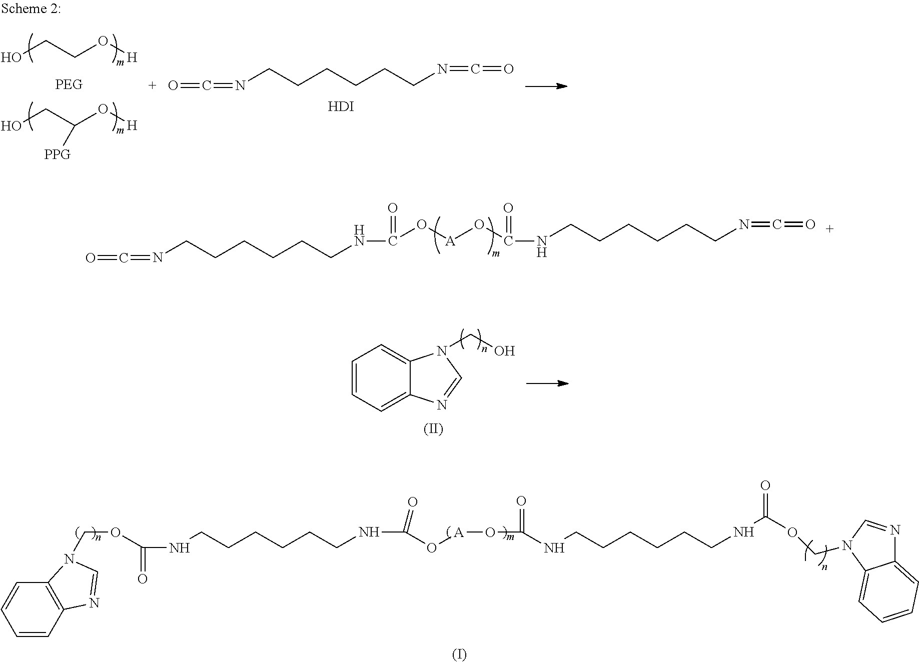

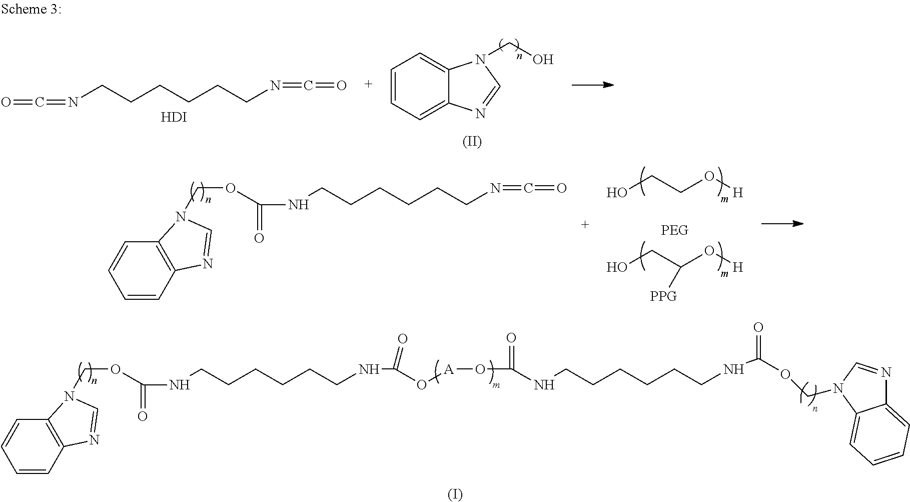

### Example 1

### Synthesis of Compound Ia-600

PEG 600 (polyethylene glycol, Mw=600) was first warmed up to 75° C., stirred, and dewatered overnight under vacuum.

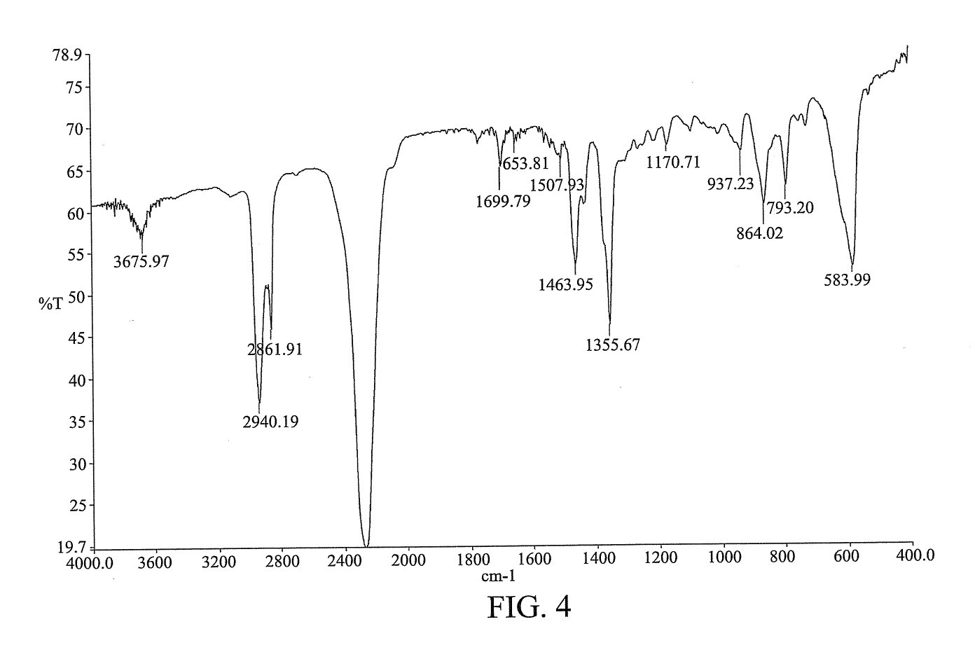

An amount of 4.64 g of PEG 600 was added to a separatory flask, stirred, warmed up to 50° C., and added rapidly with 2.86 g of HDI, The mixture was heated to 90° C. for 2 to 4 hours. (FIG. 4 shows the FTIR spectrum of HDI: C—H stretch at 2940.19, 2861.91, —NCO— stretch at 2273.23 cm¹. There is a strong absorption peak of —NCO— at about 2273.23 cm⁻¹.)

Then, the content of NCO (isocyanate group, —N═C═O) was measured by titration (according to the ASTM D2572-97 standard) to determine if the termination of the reaction of the intermediate was arrived. After the reaction was terminated, the temperature was cooled down to 75° C., 5.98 g of the intermediate was added to 2.33 g of compound (IIa), and the temperature of the mixture was maintained at 90° C. for 2 to 4 hours until the content of NCO was zero. Then, the temperature was cooled to room temperature.

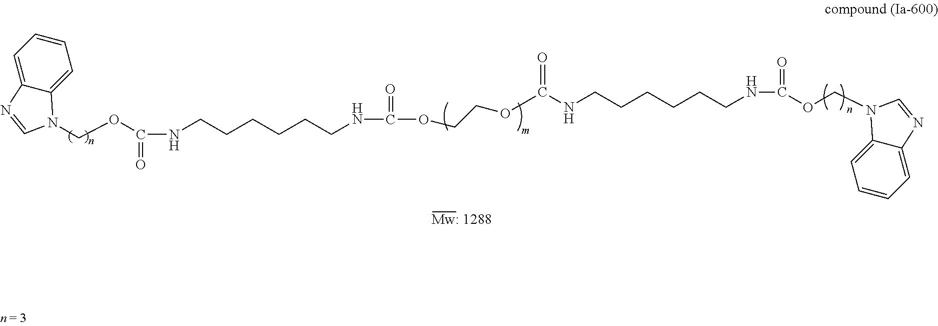

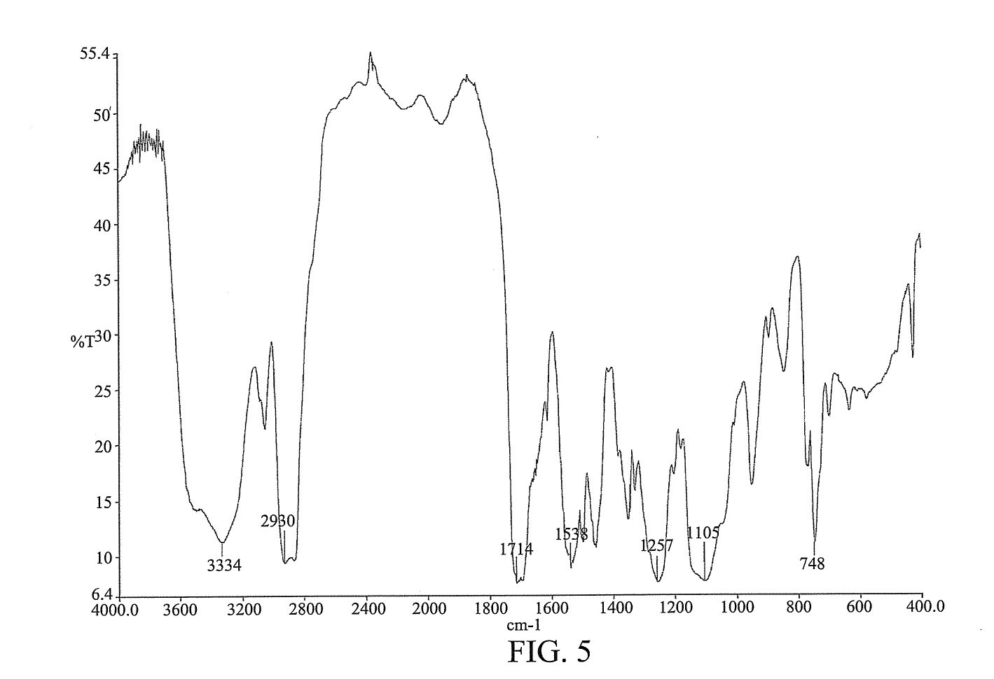

FIG. 5 shows an FTIR spectrum. (N—H at 3332.50 cm⁻¹, C═O at 1713 cm⁻¹.)

In FIG. 5, an absorption peak of —NH at about 3310 to 3500 cm⁻¹ and a very strong absorption peak of C═O at about 1700 to 1720 cm⁻¹ are shown In FIG. 5, the two absorption peaks represent the NHCOO functional group, i.e. the specific functional group of PU, which indicated that the reaction of —NCO— and —OH— took place.

### Example 2

### Synthesis of Compound Ib-600

PEG 600 was first warmed up to 75° C., stirred, and dewatered overnight under vacuum.

An amount of 4.64 g of PEG 600 was added to a separatory flask, stirred, warmed up to 50° C., and added rapidly with 2.86 g of HDI. The mixture was heated to 90° C. for 2 to 4 hours.

Then, the content of NCO was measured by titration to determine if the termination of the reaction of the intermediate was arrived. After the reaction was terminated, the temperature was cooled down to 75° C., 4.50 g of the intermediate was added to 1.72 g of compound (IIb), and the temperature of the mixture was maintained at 90° C. for 2 to 4 hours until the content of NCO was zero. Then, the temperature was cooled to room temperature.

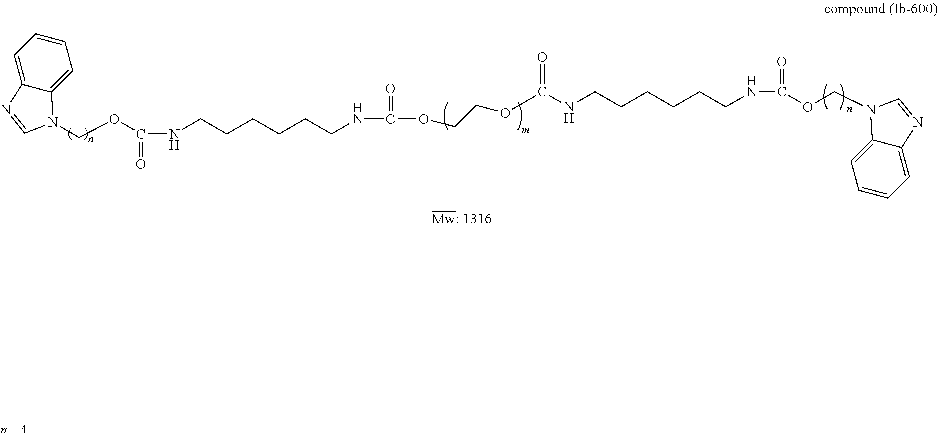

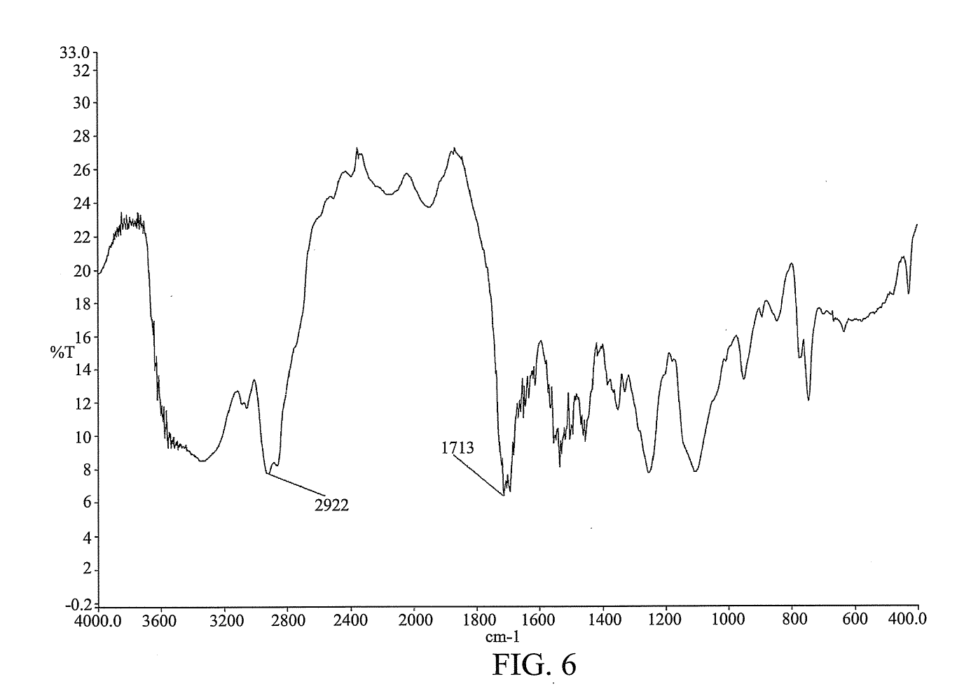

FIG. 6 shows an FTIR spectrum. (N—H at 3332.50 cm⁻¹, C═O at 1713 cm⁻¹.)

In FIG. 6, an absorption peak of —NH at about 3310 to 3500 cm⁻¹ and a very strong absorption peak of C═O at about 1700 to 1720 cm⁻¹ are shown. In FIG. 6, the two absorption peaks represent the NHCOO functional group, i.e. the specific functional group of PU, which indicated that the reaction of —NCO— and —OH— took place.

### Example 3

### Synthesis of Compound Ic-600

PEG 600 was first warmed up to 75° C., stirred, and dewatered overnight under vacuum.

An amount of 16.71 g of PEG 600 was added to a separatory flask, stirred, warmed up to 50° C., and added rapidly with 10.29 g of HDI. The mixture was heated to 90° C. for 2-4 hours.

Then, the content of NCO was measured by titration to determine if the termination of the reaction of the intermediate was arrived. After the reaction was terminated, the temperature was cooled down to 75° C., 24.73 g of the intermediate was added to 12.71 g of compound (IIc), and the temperature of the mixture was maintained at 90° C. for 2 to 4 hours until the content of NCO was zero. Then, the temperature was cooled to room temperature.

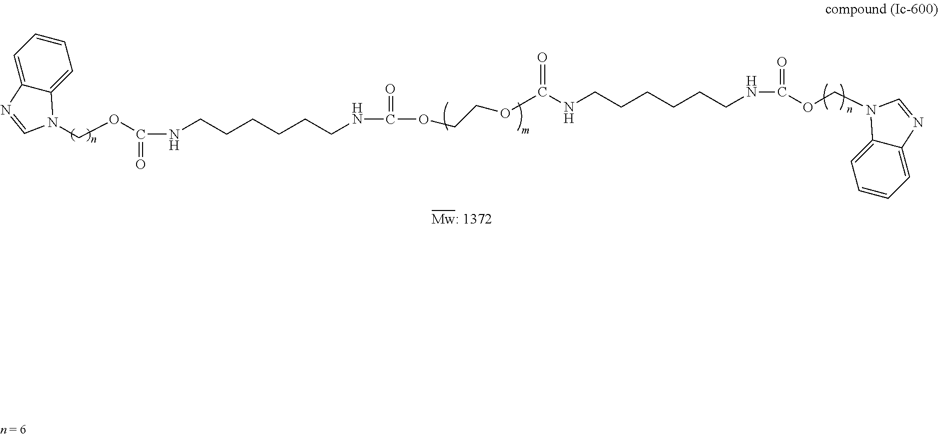

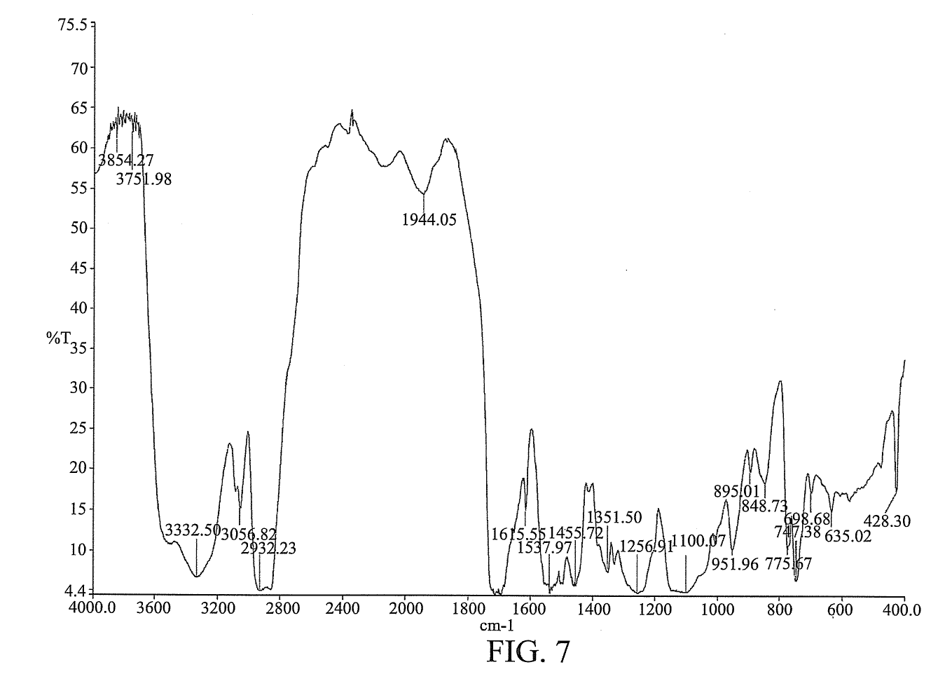

FIG. 7 shows an FTIR spectrum. (N—H at 3332.50 cm⁻¹, C═O at 1717.92 cm⁻¹.)

In FIG. 7, an absorption peak of —NH at about 3310 to 3500 cm⁻¹ and a very strong absorption peak of C═O at about 1700 to 1720 cm⁻¹ are shown. In FIG. 7, the two absorption peaks represent the NHCOO functional group, i.e. the specific functional group of PU, which indicated that the reaction of —NCO— and —OH— took place.

### Example 4

### Synthesis of Compound Ic-300

PEG 300 (polyethylene glycol, Mw=300) was first warmed up to 75° C., stirred, and dewatered overnight under vacuum.

An amount of 13.10 g of HDI was added to a separatory flask, stirred, warmed up to 50° C., and slowly added with 17.00 g of compound (IIc). The mixture was heated to 90° C. for 2 to 4 hours.

Then, the content of NCO was measured by titration to determine if the termination of the reaction of the intermediate was arrived. After the reaction was terminated, the temperature was cooled down to 75° C., 28.90 g of the intermediate was added to 11.23 g of PEG 300, and the temperature of the mixture was maintained at 90° C. for 2 to 4 hours until the content of NCO was zero. Then, the temperature was cooled to room temperature.

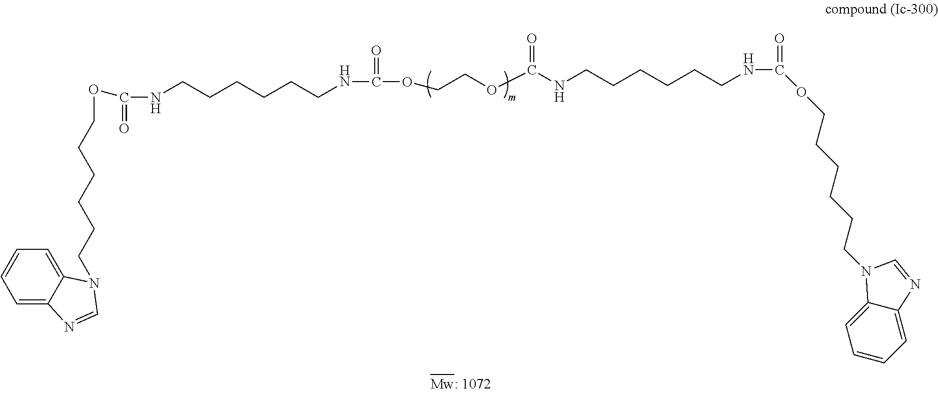

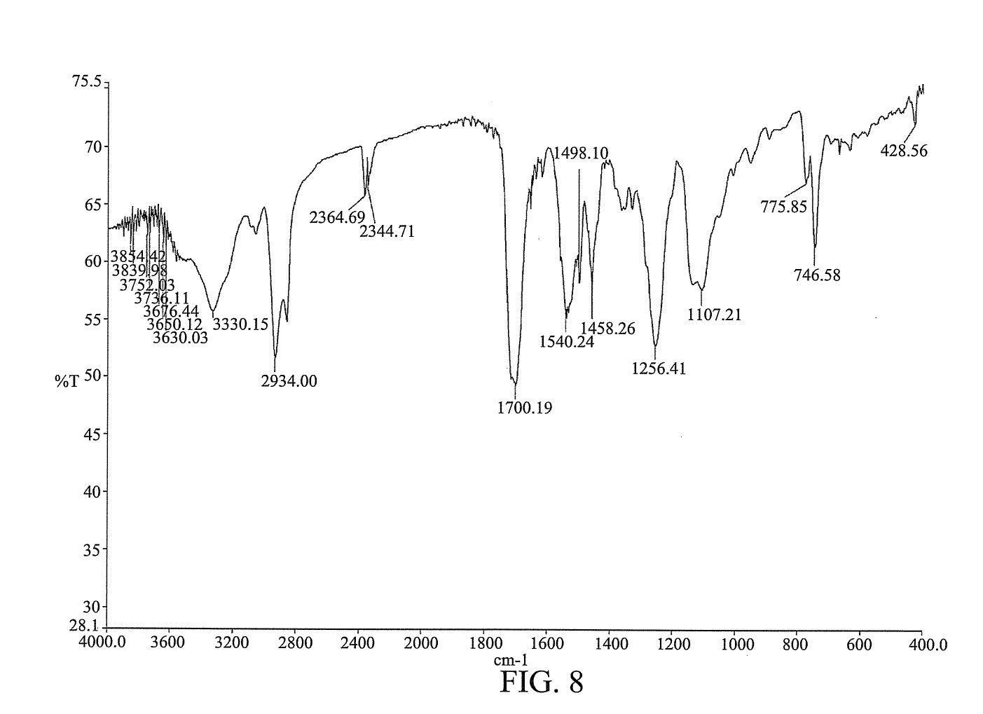

FIG. 8 shows an FTIR spectrum. (N—H at 3330.15 cm⁻¹, C═O at 1700.19 cm⁻¹.)

In FIG. 8, an absorption peak of —NH at about 3310 to 3500 cm⁻¹ and a very strong absorption peak of C═O at about 1700 to 1720 cm⁻¹ are shown. In FIG. 8, the two absorption peaks represent the NHCOO functional group, i.e. the specific functional group of PU, which indicated that the reaction of —NCO— and —OH— took place.

### Example 5

### Synthesis of Compound Ic-400

PPG 400 (polypropylene glycol, Mw=400) was first warmed up to 75° C., stirred, and dewatered overnight under vacuum.

An amount of 11.76 g of HDI was added to a separatory flask, stirred, warmed up to 50° C., and gradually added with 15.26 g of compound (IIc). The mixture was heated to 90° C. for 2-4 hours.

Then, the content of NCO was measured by titration to determine if the termination of the reaction of the intermediate was arrived. After the reaction was terminated, the temperature was cooled down to 75° C., 25.00 g of the intermediate was added to 12.95 g of PPG 400, and the temperature of the mixture was maintained at 90° C. for 2 to 4 hours until the content of NCO was zero. Then, the temperature was cooled to room temperature.

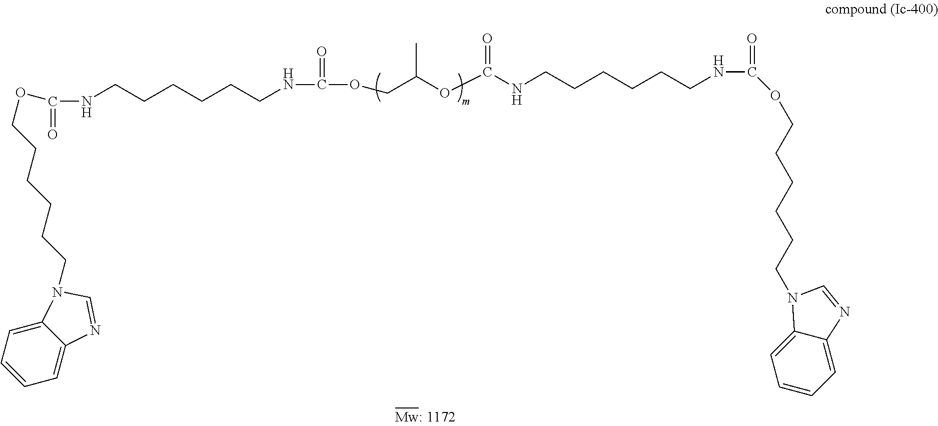

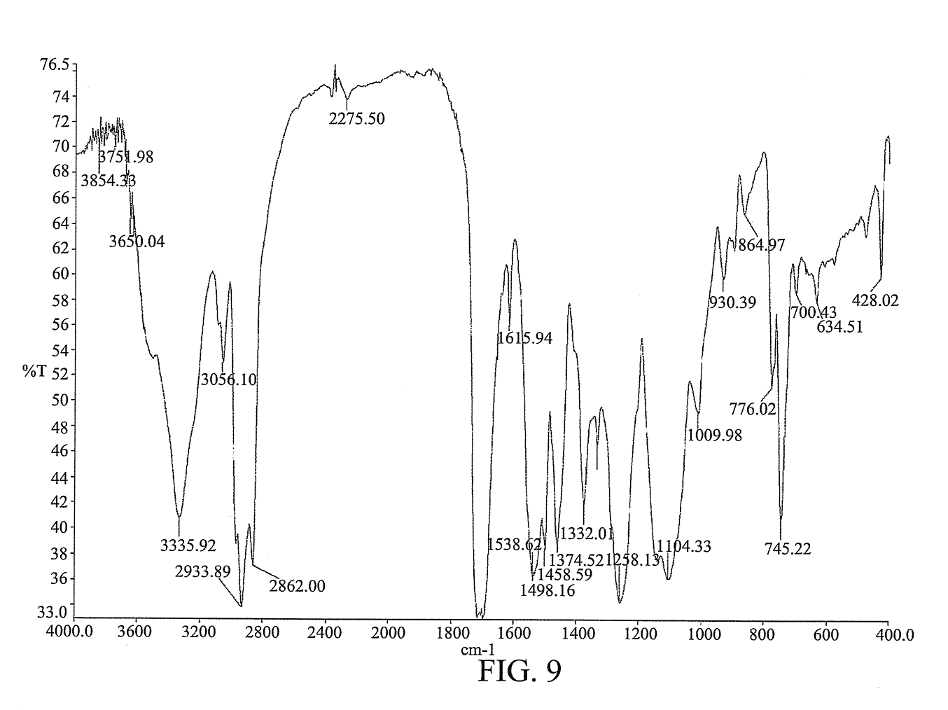

FIG. 9 shows an FTIR spectrum. (N—H at 3335.92 cm⁻¹, C═O at 1700.19 cm⁻¹.)

In FIG. 9, an absorption peak of —NH at about 3310 to 3500 cm⁻¹ and a very strong absorption peak of C═O at about 1700 to 1720 cm⁻¹ are shown. In FIG. 9, the two absorption peaks represent the NHCOO functional group, i.e. the specific functional group of PU, which indicated that the reaction of —NCO— and —OH— took place.

### Preparation of a Dye-Sensitized Solar Cell and Performance of an Efficiency Test

### Test Example 1

### Preparation of a Dye-Sensitized Solar Cell by Using a Compound of Formula (I) and Performing an Efficiency Test

A paste having titanium dioxide nanoparticles with diameters of from 20 to 30 nm was coated on a glass plate (thickness: 4 mm; resist: 105%) covered with fluorine doped tin oxide (FTO) by screen printing once or multiple times. Sintering was performed at 450° C. for 30 minutes. After sintering, the thickness of the sintered porous titanium dioxide film (i.e., the porous semiconductor film) was from 10 to 12 μm.

A dye compound (D719, Everlight) was dissolved in a mixed solution having acetonitrile and t-butanol (1:1 v/v) to form a dye solution having a dye compound in a concentration of 0.5 M. Then, the above glass plate with the porous titanium dioxide film was dipped in the dye solution to adsorb the dye compound for 16 to 24 hours. The glass plate was taken out and dried to obtain a photoanode.

The glass plate covered with FTO was primed to form a injection hole with a diameter of 0.75 mm for injecting an electrolyte therethrough. Then, the glass plate was coated with a hexachloroplatinic acid (H₂PtCl₆) solution (containing 2 mg of platinum per 1 ml of ethanol), and heated to 400° C. for 15 minutes to obtain a cathode.

A thermalplastic polymer film with a thickness of 60 μm was interposed between the photoanode and the cathode at a temperature of from 120 to 140° C., and a pressure was applied on the two electrodes to adhere them.

The electrolyte (the composition shown in Table 1) was injected through the injection hole, and the injection hole was sealed with a thermoplastic polymer film, so as to obtain a dye-sensitized solar cell.

TABLE 1
The composition of the electrolyte

| | Concentration (molar |
|---|---|
| Components | concentration, M) |
| PMII | 0.6 |
| LiI | 0.1 |
| I₂ | 0.1 |
| GuNCS | 0.1 |
| compound of formula (I) | 0.3 (in acetonitrile) |

The dye-sensitized solar cells respectively prepared from compounds (I) in Examples 1, 2, 3, 4 and 5 were tested for photo-electric conversion efficiency under the luminescence at AM1.5. The test categories included the short circuit current (J_{SC}), the open circuit voltage (V_{OC}), the photo-electric conversion efficiency (η) and the filling factor (FF). The results are shown in Table 2.

### Comparative Example 1

### An Electrolyte Free of a Compound of Formula (I)

A dye-sensitized solar cell was prepared in the same manner as in test example 1, except that the compound of formula (I) is not added in the electrolyte composition. Test results are shown in Table 2.

### Comparative Example 2

### Replacement of a Compound of Formula (I) with a Compound of Formula (IV)

A compound of formula (IV) (which was polymerized from PEG 1000 (polyethylene glycol, Mw=1000), HDI and 1-(3-aminopropyl)imidazole) replaced the compound of formula (I) in the electrolyte composition of the dye-sensitized solar cell of test example

1. Test results are shown in Table 2.

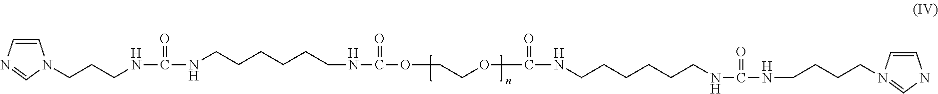

TABLE 2
An efficiency test performed on the dye-sensitized solar cell

| | | | Jsc | | |
|---|---|---|---|---|---|
| Examples | Compounds | Voc (V) | (mA) | FF (%) | η (%) |
| Test example 1 | Ia-600 (Example 1) | 0.736 | 11.72 | 64.17 | 5.54 |
| | Ib-600 (Example 2) | 0.724 | 12.9 | 61.51 | 5.74 |
| | Ic-600 (Example 3) | 0.736 | 13.00 | 61.20 | 5.86 |
| | Ic-300 (Example 4) | 0.730 | 13.82 | 63.69 | 6.43 |
| | Ic-400 (Example 5) | 0.711 | 13.68 | 62.53 | 6.08 |
| Comparative | None | 0.573 | 12.74 | 61.48 | 4.49 |
| example 1 | | | | | |
| Comparative | IV | 0.761 | 8.04 | 59.66 | 3.65 |
| example 2 | | | | | |

As shown in Table 2, the addition of compound (I) of the present invention can effectively increase the photo-electric conversion efficiency of the dye-sensitized cell.

### Test Example 2

### Preparation of a Dye-Sensitized Solar Cell from a Compound of Formula (II) and Performance of an Efficiency Test

The electrolyte (the composition shown in Table 3) was injected through the injection hole, and the injection hole was sealed with a thermalplastic polymer film, so as to obtain a dye-sensitized solar cell.

TABLE 3
The composition of the electrolyte

| | Concentration (Molar |
|---|---|
| Components | concentration, M) |
| PMII | 0.6 |
| LiI | 0.1 |
| I₂ | 0.1 |
| GuNCS | 0.1 |
| compound of formula (II) | 0.3 (in 3-MPN) |

The dye-sensitized solar cells respectively prepared from compounds (IIa), (IIb) and (IIc) in Synthesis Examples 1-3 were tested for photo-electric conversion efficiency under the luminescence at AM 1.5. The test categories included the short circuit current (J_{SC}), the open circuit voltage (V_{OC}), the photo-electric conversion efficiency (η) and the filling factor (FF). The results are shown in Table 4.

### Comparative Example 3

### Replacement of the Compound of Formula (II) in the Composition of the Electrolyte in Test Example 2 with N-Butylbenzimidazole (NBB) and Performance of a Test

TABLE 4
Efficiency test performed on the dye-sensitized solar cell

| | | | | | η |
|---|---|---|---|---|---|
| example | Compound | Voc | Jsc | FF (%) | (%) |
| Test example 2 | IIa (Synthesis example 1) | 0.778 | 9.17 | 62.62 | 4.46 |
| | IIb (Synthesis example 2) | 0.782 | 8.85 | 60.69 | 4.2 |
| | IIc (Synthesis example 3) | 0.768 | 9.64 | 61.13 | 4.52 |
| Comparative | NBB | 0.746 | 8.99 | 62.01 | 4.16 |
| example 3 | | | | | |

As shown in Table 4, compound (II) of the present invention can prevent dark currents and enhance an increase in an open circuit voltage (V_{OC}). Further, the compound (II) of the present invention may increase the photo-electric conversion efficiency of a dye-sensitized solar cell.

The compounds of formulae (I) and (II) of the present invention can be used in an electrolyte for a dye-sensitized solar cell. The electrolyte containing the compounds of the present invention can be used to prevent dark currents and enhance an increase in an open circuit voltage (V_{OC}). Further, the addition of the compounds of formulae (I) and/or (II) can increase the photo-electric conversion efficiency of a dye-sensitized solar cell, and thereby meeting the industrial requirement.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

1. A compound of formula (I): 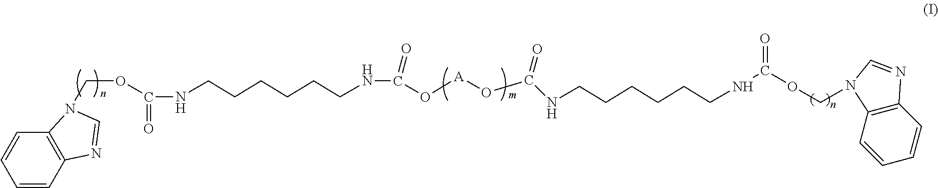
wherein A is C₂₋₃ alkylene; m is an integer ranging from 2 to 25; and n is an integer ranging from 3 to 10.

2. The compound of claim 1, wherein A is ethylene, and m is an integer ranging from 2 to 25.

3. The compound of claim 1, wherein A is isopropylene, and m is an integer ranging from 2 to 15.

4. The compound of claim 1, wherein n is an integer ranging from 3 to 8.

5. The compound of claim 4, wherein n is an integer ranging from 3 to 6.

6. The compound of claim 1, which is used in an electrolyte for a solar cell.

7. The compound of claim 6, which is used in an electrolyte for a dye-sensitized solar cell.

8. An electrolyte for a dye-sensitized solar cell, comprising the compound of formula (I) of claim 1 or a compound of formula (II): 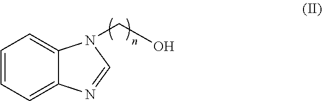
wherein n is an integer ranging from 3 to 10.

9. The electrolyte for a dye-sensitized solar cell of claim 8, wherein n is an integer ranging from 3 to 8.

10. The electrolyte for a dye-sensitized solar cell of claim 9, wherein n is an integer ranging from 3 to 6.

11. The electrolyte for a dye-sensitized solar cell of claim 8, further comprising at least one selected from a group consisting of metal iodide, an imidazolium iodide salt derivative and a salt of a combination thereof, iodine, guanidine thiocyanate, and a solvent.

12. A dye-sensitized solar cell, comprising:
a substrate;
a porous semiconductor film;
a conductive film;
the electrolyte for a dye-sensitized solar cell of claim 8; and
a dye compound.

13. The dye-sensitized solar cell of claim 12, wherein the electrolyte further comprises at least one selected from a group consisting of metal iodide, an imidazolium iodide salt derivative and a salt of a combination thereof, iodine, guanidine thiocyanate, and a solvent.

14. A method for preparing the compound of claim 1, comprising the steps of:
performing a reaction of polyallylene glycol, hexamethylene diisocyanate (HDI) and a compound of formula (II) to form polyurethane, wherein the compound of formula (II) is: 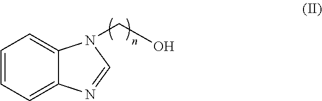
wherein n is an integer ranging from 3 to 10.

15. The method of claim 14, wherein the polyalkylene glycol is one of polyethylene glycol and polypropylene glycol.

16. The method of claim 15, wherein the polyalkylene glycol is polyethylene glycol having a molecular weight ranging from 100 to 1000.

17. The method of claim 15, wherein the polyalkylene glycol is polypropylene glycol having a molecular weight ranging from 200 to 1000.
